(19) United States
(12) Patent Application Publication
(10) Pub. No.: US 2023/0331853 A1
(43) Pub. Date: Oct. 19, 2023

(54) METHODS AND COMPOSITIONS FOR TREATING RESPIRATORY DISEASES OR CONDITIONS RELATED TO INNATE LYMPHOID CELLS

(71) Applicant: NATIONAL JEWISH HEALTH, Denver, CO (US)

(72) Inventors: R. Lee REINHARDT, Denver, CO (US); Mindy MILLER, Lakewood, CO (US)

(21) Appl. No.: 18/044,349

(22) PCT Filed: Sep. 10, 2021
(86) PCT No.: PCT/US21/49909

§ 371 Date: Mar. 7, 2023

Related U.S. Application Data
(60) Provisional application No. 63/077,345, filed on Sep. 11, 2020.

(51) Int. Cl. C07K 16/28; A61P 37/06

(52) U.S. Cl. CPC C07K 16/2851; A61P 37/06; A61K 2039/505

(57) ABSTRACT

Disclosed herein are methods and compositions for treating Group 2 innate lymphoid cell (ILC2) mediated diseases or conditions comprising administering a killer cell lectin-like receptor G1 (KLRG1) binding agent or KLRG1 ligand binding agent or antagonist thereof.

### CROSS-REFERENCE TO RELATED APPLICATIONS

This application claims the benefit of priority under 35 U.S.C. § 119(e) to U.S. Provisional Patent Application No. 63/077,345, filed Sep. 11, 2020. The entire disclosure of U.S. Provisional Patent Application No. 63/077,345 is incorporated herein by reference.

### GOVERNMENT SUPPORT

This invention was made with government support under grant number R01 AI119004 awarded by the National Institutes of Health. The government has certain rights in the invention.

### BACKGROUND

Killer cell lectin-like receptor G1 (KLRG1) is a 30-38 kDa type-II transmembrane C-type lectin glycoprotein. This molecule is highly conserved across mammals. KLRG1 is expressed as both a monomer and homodimer at the cell surface. The ligands for KLRG1 are type I cadherins. Cadherins are adhesion molecules that help to bind cells together at cell-to-cell junctions. In addition to their adhesion properties, cadherins are also described as mechano-transducers that sense changes in intercellular junction integrity. KLRG1 can bind classical type 1 cadherins (epithelial (E), neuronal (N), and retinal (R)). Although E-cadherin is enriched at epithelial junctions, it is also expressed on macrophages, dendritic cells, and osteoclasts. Similarly, N-cadherin is found in both neuronal and vascular tissue suggesting a broader pattern of expression beyond neurons.

Functionally, KLRG1 is often classified as an inhibitory receptor due to the presence of an immune receptor tyrosine-based inhibitory motif (ITIM) in the cytoplasmic tail of the molecule. ITIM domains contain phosphorylation sites that recruit tyrosine phosphatases that dampen cellular signaling pathways in Natural Killer (NK) cells and CD8 T cells (specifically effector and effector memory subsets). In vitro, KLRG1 engagement leads to diminished cell-mediated cytotoxicity, cytokine release, and T cell receptor-mediated proliferation. KLRG1 expression on NK cells prevents killing of E-cadherin expressing target cells, and KLRG1+ expression on CD8 T cells is a marker of activation. Upon repeated antigen exposure, KLRG1 also serves as a marker of cellular senescence among responding CD8+ T cells. Together, these findings have implicated KLRG1 in tumor surveillance and killing of metastatic epithelial tumors (i.e. breast cancer) which are associated with decreased E-cadherin expression. Although targeting KLRG1 is being explored as a target in cancer therapy, its role in other disease settings such as those induced by type-2 inflammation (allergic disease, helminth infection, etc.) remains unknown.

Although classically defined as a receptor on NK cells and subsets of CD8 T cells, KLRG1 is also expressed by CD4+ T cells, regulatory T cells (Tregs) and group 2 innate lymphoid cells (ILC2s). Despite evidence of high expression of KLRG1 on both human and mouse ILC2 cells, the role of KLRG1 in modulating ILC2 cell behavior and function is largely undefined.

### BRIEF DESCRIPTION OF THE DRAWINGS

FIGS. 1A-1H show the gating strategy for pulmonary iILC2 cells. Yetcre13-YFP reporter mice were infected with N. brasiliensis and five days later lung cells were processed for flow cytometry. ILC2 cells are identified as live, low forward- and side-scatter, singlet, CD45 positive, Lineage negative, CD90 positive, and CD127 positive. iILC2s can be further delineated as CD90 low and KLRG1 high (FIG. 1F), and IL-13 cytokine-producing iILC2 cells can be identified by expression of the YFP reporter (yetcre13) along with IL17RB expression (FIG. 1G) or low CD90 expression FIG. 1H). The lineage antibody cocktail includes antibodies against CD3, CD4, CD11b, CD11c, B220, NK1.1, FcεR1a, GR-1, DX5, TCRβ, γδTCR, and Ter119.

FIGS. 2A-2B shows that in-vivo anti-KLRG1 antibody treatment results in complete blocking of KLRG1 epitopes during flow cytometry analysis. Flow cytometry plots of mouse lung cells 5 days after N. brasiliensis infection. The plot in FIG. 2A shows a mouse that received no anti-KLRG1 treatment. The plot in FIG. 2B is from a mouse given daily intravenous 100 ug anti-KLRG1 injections 2, 3, and 4 days after infection. The data is representative of 7 mice per treatment compiled from 2 separate experiments.

FIGS. 3A-3D show that anti-KLRG1 treatment significantly reduces the number and frequency of iILC2 cells in the lung after helminth infection. Mice were infected with N. brasiliensis and either left untreated or given intravenous 100 ug anti-KLRG1 antibody 2, 3, and 4 days later. On day 5, lungs were harvested and processed for flow cytometry analysis of iILC2s shown as CD90-low IL17RB-positive.

FIGS. 4A-4D show that anti-KLRG1 treatment significantly reduces IL-13-producing iILC2s after helminth infection. Yetcre13-YFP reporter mice were infected with N. brasiliensis and either left untreated or given intravenous 100 ug anti-KLRG1 antibody 2, 3, and 4 days later. On day 5, lungs were harvested and processed for flow cytometry analysis of IL-13 (YFP)-producing IL17RB-positive iILC2s.

FIGS. 5A-5G shows the gating strategy for pulmonary eosinophils. Mice were infected with N. brasiliensis and five days later lung cells were processed for flow cytometry. Eosinophils are identified as live, mid forward- and high side-scatter, singlet, CD4 negative, B220 negative, Siglec-F positive, DX5 negative, and CD11b positive. Tissue resident eosinophils (rEos) are further identified as CD11c negative whereas inflammatory eosinophils (iEos) are CD11c positive.

FIGS. 6A-6G show that anti-KLRG1 treatment reduces the recruitment of inflammatory eosinophils to the lung after helminth infection. Yetcre13-YFP reporter mice were left uninfected or infected with N. brasiliensis and either left untreated or given intravenous 100 ug anti-KLRG1 antibody 2, 3, and 4 days later. On day 5, lungs were harvested and processed for flow cytometry analysis of eosinophil subsets.

### SUMMARY

One embodiment relates to a method of treating a Group 2 innate lymphoid cell (ILC2) mediated disease comprising administering to a subject in need thereof an effective amount of a killer cell lectin-like receptor G1 (KLRG1) binding agent or KLRG1 ligand binding agent.

In one aspect, the binding agent is an antibody or antigen binding fragment thereof, an antibody mimetic or an antibody mimetic that binds the extracellular domain of human KLRG1.

In one aspect, the antibody or antigen binding fragment thereof, or antibody mimetic comprises a human or humanized antibody. In yet another aspect, the antibody or antigen binding fragment thereof, or antibody mimetic comprises: a full length antibody Fab portion that binds KLRG1; b. a full length antibody Fab portion that binds E-cadherin; c. a full length antibody Fab portion that binds N-cadherin; d. a full length antibody Fab portion that binds R-cadherin; e. a fusion protein E-cadherin/Fc; f. a fusion protein R-cadherin/Fc; g. a fusion protein N-cadherin/Fc; h. a chimeric antigen receptor; or i. a multi-specific antibody. In one aspect, the multi-specific antibody comprises a bispecific or tri-specific antibody.

In one aspect, the binding agent binds KLRG1.

In another aspect, the KLRG1 is the extracellular domain of human KLRG1.

In one aspect, the binding agent binds KLRG1 ligand.

In one aspect, the KLRG1 ligand is human E-cadherin, N-cadherin, or R-cadherin.

In still another aspect, the binding agent binds KLRG1 and/or cadherin at a KLRG1/cadherin binding site.

Another embodiment relates to a method of treating a Group 2 innate lymphoid cell (ILC2) mediated disease in a subject in need thereof comprising administering to the subject in need thereof an effective amount of a KLRG1 antagonist or a KLGR1 ligand antagonist.

In one aspect, the KLRG1 antagonist comprises (i) KLRG1 binding agent or KLRG1 ligand binding agent or (ii) an RNAi agent that suppresses KLRG1 expression or (iii) an RNAi agent that suppresses the KLRG1 ligand expression.

In one aspect, the KLRG1 antagonist disrupts KLRG1 signaling, thereby activating CD8⁺ cytotoxic T and/or NK cells.

In still another aspect, the KLRG1 antagonist binds the extracellular domain of human KLRG1 or binds a KLRG1 ligand.

In yet another aspect, the KLRG1 antagonist comprises an antibody or antigen binding fragment. In one aspect, the antibody is monoclonal. In another aspect, the antibody is human or humanized.

In still another aspect, the KLRG1 antagonist comprises an Fc-cadherin fusion protein.

In another aspect, the KLRG1 antagonist comprises a binding agent that blocks or competes with KLRG1-ligand binding

In one aspect, the KLRG1 antagonist comprises a binding agent that cross reacts with the extracellular domains of human KLRG1, or the human KLRG1 ligand

In still another aspect, the KLRG1 antagonist comprises a binding agent that binds to KLRG1 and that is not a mouse antibody

In yet another aspect, the KLRG1 antagonist comprises an RNAi agent, an siRNA or an mRNA.

In any of the above aspects, the ILC2 mediated disease is selected from the group consisting of asthma, atopic dermatitis, allergic airway disease, airway hyperreactivity, idiopathic pulmonary fibrosis, chronic rhinosinusitis, Respiratory syncytial virus induced airway inflammation, chronic obstructive pulmonary disease (COPD), liver fibrosis, viral hepatitis, atherosclerosis, multiple sclerosis, Eosinophilic esophagitis. In one aspect, the asthma is fungal asthma or viral asthma from influenza A. In yet another aspect, the allergic airway disease is allergic rhinitis, allergic dermatitis or allergic inflammation from ragweed, house dust mite, or protease allergens.

Another embodiment relates to a killer cell lectin-like receptor G1 (KLRG1) antagonist comprising (i) a killer cell lectin-like receptor G1 (KLRG1)/ligand binding agent or (ii) an RNAi agent that suppresses KLRG1 expression.

In one aspect, the antagonist disrupts KLRG1 signaling, thereby activating CD8⁺ cytotoxic T and/or NK cells.

In still another aspect, the antagonist binds the extracellular domain of human KLRG1 or binds a KLRG1 ligand.

In yet another aspect, the KLRG1 antagonist comprises an antibody or antigen binding fragment. In one aspect, the antibody is monoclonal. In yet another aspect, the antibody is human or humanized.

In another aspect, the KLRG1 antagonist comprises an Fc-cadherin fusion protein.

Another embodiment is an isolated KLGR1 monoclonal antibody.

Another embodiment is a nucleic acid polymer encoding an isolated KLGR1 monoclonal antibody.

### DETAILED DESCRIPTION

The invention is based, at least in part, on the discovery that although classically defined as a receptor on NK cells and subsets of CD8 T cells, KLRG1 is also expressed by CD4+ T cells, regulatory T cells (Tregs) and group 2 innate lymphoid cells (ILC2s). Despite evidence of high expression of KLRG1 on both human and mouse ILC2 cells, the role of KLRG1 in modulating ILC2 cell behavior and function is largely undefined. The data generated and disclosed herein shows that targeting of KLRG1 during helminth infection (a model of robust type-2 inflammation) inhibits early accumulation of migratory ILC2 cells to the lung and limits ILC2-mediated type-2 cytokine production. The data also shows that targeting KLRG1 can prevent eosinophilia and pathology associated with type-2 inflammation in the lung. This is the first evidence that targeting of KLRG1 can limit type-2 inflammation indicting that KLRG1 can have therapeutic value in ameliorating allergic disease and diseases associated with ILC2 function.

Accordingly, disclosed herein are methods of treating a Group 2 innate lymphoid cell (ILC2) mediated disease comprising administering to a subject in need thereof an effective amount of a killer cell lectin-like receptor G1 (KLRG1) binding agent or KLGR1 ligand binding agent. The invention also provides methods of treating an ILC2 mediated disease in a subject in need thereof comprising administering to a subject in need thereof an effective amount of a KLRG1 antagonist and/or a KLGR1 ligand antagonist.

The KLGR1 binding agent is an antibody or antigen binding fragment thereof, an antibody mimetic or an antibody mimetic that binds the extracellular domain of human KLRG1. The antibody or antigen binding fragment thereof, or antibody mimetic can be a human or humanized antibody. The antibody or antigen binding fragment thereof, or antibody mimetic can comprise a full length antibody Fab portion that binds KLRG1; a full length antibody Fab portion that binds E-cadherin; a full length antibody Fab portion that binds N-cadherin; a full length antibody Fab portion that binds R-cadherin; a fusion protein E-cadherin/Fc; a fusion protein R-cadherin/Fc; a fusion protein N-cadherin/Fc; a chimeric antigen receptor; or a multi-specific antibody. The multi-specific antibody can be a bispecific or tri-specific antibody. Further, the binding agent is an agent that binds to KLGR1.

KLRG1 is type II transmembrane protein surface co-inhibitory receptor modulating the activity of T and NK cells. The binding agent as disclosed herein can be an agent that binds to KLGR1 or binds to a KLGR1 ligand. KLGR1 has an extracellular portion containing a C-type lectin domain whose known ligands are cadherins and its intracellular portion that contains an immunoreceptor tyrosine-based inhibitory motif (ITIM) domain responsible for co-inhibition of T cell receptor (TCR) mediated signaling (Tessmer et al. “KLGR1 binds cadherins and preferentially associates with SHIP-1”; Int Immunol. Apr;19(4):391-400; 2007). In various embodiments, the ligand can be human E-cadherin, N-cadherin, R-cadherin, or a combination thereof. In addition, it is contemplated herein that the binding agent can bind KLRG1 and/or cadherin at a KLRG1/cadherin binding site.

A killer cell lectin-like receptor G1 (KLRG1) antagonist comprises (i) a killer cell lectin-like receptor G1 (KLRG1)/ligand binding agent or (ii) an RNAi agent that suppresses KLRG1 expression. The binding agent is an antibody or antigen binding fragment thereof, or antibody mimetic. In general, the antagonist disrupts KLRG1 signaling, thereby achieving a therapeutic effect (e.g., by activating CD8⁺ cytotoxic T and/or NK cells). The antagonist binds the extracellular domain of human KLRG1 or binds a KLRG1 ligand (e.g., thus disrupting KLRG1 signaling). The antagonist can comprise an Fc-cadherin fusion protein. Further the antagonist can comprise a binding agent that blocks or competes with KLRG1-ligand binding. The KLRG1 antagonist can comprise a binding agent that cross reacts with the extracellular domains of human KLRG1, or the human KLRG1 ligand.

In various aspects, the KLGR1 antagonist comprises an antibody or antigen binding fragment. The antibody can be monoclonal. Further, the antibody can be human or humanized. the KLRG1 antagonist comprises a binding agent that binds to KLRG1 and that is not a mouse antibody.

In various aspects, the KLGR1 antagonist comprises an RNAi agent, an siRNA or an mRNA.

The ILC2 mediated disease is a disease selected from the group consisting of asthma, atopic dermatitis, allergic airway disease, airway hyperreactivity, idiopathic pulmonary fibrosis, chronic rhinosinusitis, respiratory syncytial virus induced airway inflammation, chronic obstructive pulmonary disease (COPD), liver fibrosis, viral hepatitis, atherosclerosis, multiple sclerosis, Eosinophilic esophagitis. In one aspect, the asthma is fungal asthma or viral asthma from influenza A. In another aspect, the allergic airway diseases is allergic rhinitis, allergic dermatitis or allergic inflammation from ragweed, house dust mite, or protease allergens.

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

It is further understood that reference to a peptide, a polypeptide or a protein herein, such as an antibody or a fragment thereof, includes pharmaceutically acceptable salts thereof unless specifically stated otherwise or the context clearly indicates otherwise. Such salts can have a positive net charge, a negative net charge or no net charge.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Unless defined otherwise or clearly indicated otherwise by their use herein, all technical and scientific terms used herein have the same meaning as commonly understood by those of ordinary skill in the art to which this application belongs.

As used in the specification and the appended claims, the indefinite articles “a” and “an” and the definite article “the” can include plural referents as well as singular referents unless specifically stated otherwise or the context clearly indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within ±10%, 5%, 4%, 3%, 2% or 1% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

The term “antibody” refers to a protein functionally defined as a binding protein and structurally defined as comprising an amino acid sequence that is recognized as being derived from the framework region of an immunoglobulin encoding gene. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “antibody” is used in the broadest sense and specifically covers, for example, single anti-KLRG1 or anti-KLRG1 ligand monoclonal antibodies. The term “monoclonal antibody” as used herein refers to an antibody obtained from a population of substantially homogeneous antibodies, e.g., the individual antibodies comprising the population are identical except for possible naturally-occurring mutations that may be present in minor amounts. An antibody can be monoclonal. An antibody can be a human or humanized antibody.

A typical gamma immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well-characterized fragments. Thus, for example, pepsin digests an antibody below the disulfide linkages in the hinge region to produce F(ab)′₂, a dimer of Fab′ which itself is naturally a light chain joined to VH-CH1-Hinge by a disulfide bond. The F(ab)′₂ may be reduced under mild conditions to break the disulfide linkage/s in the hinge region thereby converting the (Fab′)₂ dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill in the art will appreciate that fragments can be synthesized de novo either chemically or by utilizing recombinant DNA methods. Thus, the term antibody, as used herein also includes antibody fragments either produced by the modification of whole antibodies or synthesized using recombinant DNA methodologies. Preferred antibodies include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988, which is hereby incorporated by reference in its entirety). While the V_{H} and V_{L} are connected to each as a single polypeptide chain, the V_{H} and V_{L} domains associate non-covalently. Alternatively, the antibody can be another fragment. Other fragments can also be generated, including using recombinant techniques. For example Fab molecules can be displayed on phage if one of the chains (heavy or light) is fused to g3 capsid protein and the complementary chain exported to the periplasm as a soluble molecule. The two chains can be encoded on the same or on different replicons; the two antibody chains in each Fab molecule assemble post-translationally and the dimer is incorporated into the phage particle via linkage to one of the chains of g3p (see, e.g., U.S. Pat. No: 5,733,743, which is hereby incorporated by reference in its entirety). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778, all of which are hereby incorporated by reference in their entirety). Particularly preferred antibodies include all those that have been displayed on phage or generated by recombinant technology using vectors where the chains are secreted as soluble proteins, e.g., scFv, Fv, Fab, (Fab′)₂. Antibodies can also include diabodies and minibodies. “Antibody fragments” can include a portion of an intact antibody, preferably the antigen binding or variable region of the intact antibody. Examples of antibody fragments include Fab, Fab′, F(ab′)2, and Fv fragments, diabodies, linear antibodies, single-chain antibody molecules, and multispecific antibodies formed from antibody fragments.

“Fv” includes the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

Antibodies also include heavy chain dimers, such as antibodies from camelids. Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called V_{HH} domains.

In camelids, the diversity of antibody repertoire is determined by the complementary determining regions (CDR) 1, 2, and 3 in the V_{H} or V_{HH} regions. The CDR3 in the camel V_{HH} region is characterized by its relatively long length averaging 16 amino acids (Muyldermans et al., 1994, Protein Engineering 7(9): 1129, which is hereby incorporated by reference in its entirety). This is in contrast to CDR3 regions of antibodies of many other species. For example, the CDR3 of mouse V_{H} has an average of 9 amino acids.

Libraries of camelid-derived antibody variable regions, which maintain the in vivo diversity of the variable regions of a camelid, can be made by, for example, the methods disclosed in U.S. Patent Application publication No. US20050037421, published Feb. 17, 2005, which is hereby incorporated by reference in its entirety.

Antibody-dependent cell-mediated cytotoxicity (ADCC) is an important mechanism of action of antibodies. ADCC may be enhanced by several methods, many of which involve an end product antibody with improved Fc-receptor binding. Amino acid substitutions in the antibody Fc region have been shown to increase Fc binding affinity for Fc [symbol] RIIIa receptor on NK cells (Natsume et al., Drug Design, Development and Therapy, 2009, vol. 3, pp. 7-16, which is hereby incorporated by reference in its entirety) and to improve ADCC activity. Another method for improving ADCC is to change the sugar composition of the antibody glycosylation. This is done by making antibodies that lack fucose residues, which is to say, an ‘a-fucosylated’ or ‘de-fucosylated’ antibody. One method involves changing/modifying the glycosylation site of the antibody so that fucose cannot be added to the antibody (U.S. Pat. No. 6,194,551, Feb. 27, 2001). Another method is to remove a pre-existing fucose on an antibody by, for example, enzymatic degradation or removal of the fucose by any other means. Another method involves the genetic engineering of the host expression system so that fucose cannot be transferred to or added to the antibody, for example by suppression or deletion of fucosyl transferase activity (U.S. Patent Application publication Nos.: 20070134759. Jun. 14, 2007; and 20080166756, Jul. 10, 2008, both of which are hereby incorporated by reference in their entirety).

As used herein, the term “antigen” refers to substances that are capable, under appropriate conditions, of inducing a specific immune response and of reacting with the products of that response, such as, with specific antibodies or specifically sensitized T-lymphocytes, or both. Antigens may be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” may be used to refer to any substance to which an antibody binds, or for which antibodies are desired, regardless of whether the substance is immunogenic. For such antigens, antibodies may be identified by recombinant methods, independently of any immune response.

In various embodiments, the antibody or antigen binding fragment thereof, or antibody mimetic comprises a human or humanized antibody. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. Methods for humanizing non-human antibodies are well known in the art.

The binding agents may also be affinity matured, for example using selection and/or mutagenesis methods known in the art. In general, an “affinity matured” antibody is one with one or more alterations in one or more hyper variable regions thereof which result in an improvement in the affinity of the antibody for antigen, compared to a parent antibody which does not possess those alteration(s). In one embodiment, an affinity matured antibody has nanomolar or even picomolar affinities for the target antigen. Preferred affinity matured antibodies have an affinity which is five times, more preferably 10 times, even more preferably 20 or 30 times greater than the starting antibody (generally murine, humanized or human) from which the matured antibody is prepared.

As used herein, the term “binding specificity” of an antibody refers to the identity of the antigen to which the antibody binds, preferably to the identity of the epitope to which the antibody binds. An antibody that “binds to,” “specifically binds to,” or is “specific for” a particular polypeptide or an epitope on a particular polypeptide is one that binds to that particular polypeptide or epitope on a particular polypeptide without substantially binding to any other polypeptide or polypeptide epitope. As such, a KLRG1/ligand binding agent includes functional equivalents to an anti-KLRG1/ligand antibody according to the invention. A KLRG1/ligand binding agent can be a binding agent that binds to or specifically binds to KLRG1 (e.g., human KLRG1). In some cases, the KLRG1/ligand binding agent may be cross reactive with various similar KLRG1 proteins (e.g., with highest affinity for one, such as human HLRG1, and lower affinity for others, such as mouse KLRG1). A KLRG1/ligand binding agent can be a binding agent that binds to or specifically binds to a KLRG1 ligand. Again, the KLRG1/ligand binding agent can be a binding agent that binds to or specifically binds to one, or in some cases cross reacts with more than one, KLRG1 ligand (e.g., one or more human cadherins).

As used herein, the term “chimeric polynucleotide” means that the polynucleotide comprises regions which are wild-type and regions which are mutated. It may also mean that the polynucleotide comprises wild-type regions from one polynucleotide and wild-type regions from another related polynucleotide.

As used herein, the term “complementarity-determining region” or “CDR” refer to the art-recognized term as exemplified by Kabat and Chothia. CDRs are also generally known as hypervariable regions or hypervariable loops (Chothia and Lesk (1987) J Mol. Biol. 196: 901; Chothia et al. (1989) Nature 342: 877; E. A. Kabat et al., Sequences of Proteins of Immunological Interest (National Institutes of Health, Bethesda, Md.) (1987); and Tramontano et al. (1990) J Mol. Biol. 215: 175, all of which are hereby incorporated by reference in their entirety). “Framework region” or “FR” refers to the region of the V domain that flank the CDRs. The positions of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. Jan 1;29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996, all of which are hereby incorporated by reference in their entirety).

In various aspects, the binding agent is a blocking or antagonist binding agent. “Blocking” or “antagonist” means the agent (e.g., antibody or binding fragment/mimic thereof) is one which inhibits or reduces biological activity of the antigen it binds. Certain blocking agents or antagonist agents substantially or completely inhibit the biological activity of the antigen. For example, a KLRG1/ligand binding agent can block KLRG1 signaling (e.g., thereby disrupting KLRG1 signaling and activating CD8⁺ cytotoxic T and/or NK cells). In various embodiments, the binding agent is a human or humanized monoclonal antibody, or binding fragment thereof.

In various aspects, the binding agent is not a prior art antibody. Known anti-KLRG1 antibodies include clone 13F12F2 (eBioscience), which is a mouse anti-human KLRG1 antibody that binds to the extracellular domain and has demonstrated reactivity against human cells in flow cytometry, clones 14C2A07 (Biolegend) and SA231A2 (Biolegend), which are reported to be anti-human KLRG1 antibodies, and clone 2F1, which is a hamster anti-mouse KLRG1 antibody that some vendors (e.g., Biolegend) report to be reactive against human while others (e.g., Abcam) report reactivity to only mouse. Tests of these antibodies failed to demonstrate reactivity to human KLRG1. Clone 13A2 (EBioscience) is said to bind a similar epitope to clone 13F12F2. Clone REA261 (Miltenyi Biotec) also reportedly binds human KLRG1. (Binding not verified.) Another clone is Clone ABC-m01 (Abcuro, Inc.) Known anti-E-cadherin antibodies are described by vendors and include the following examples: clone 67A4, clone MB2, and clone HECD1 (all sold by Abcam); DECMA1 sold by eBioscience; and clone 36/E-cadherin sold by BD Biosciences. In various embodiments, the KLRG1 antagonist comprises a binding agent that binds to KLRG1 and that is not clone 13F12F2, 14C2A07, SA231A2, 2F1, 13A2, or REA261 (or another previously described anti-KLRG1 antibody).

The KLRG1 antagonist comprises a binding agent that blocks or competes with KLRG1-ligand binding. In some aspects, the KLRG1 antagonist comprises a binding agent that binds to KLRG1 and that is not a mouse antibody. In various embodiments, the antagonist neutralizes KLRG1/E-cadherin binding. In various embodiments, the KLRG1 antagonist comprises an RNAi agent. The RNAi agent can be, inter alia, an siRNA or an mRNA.

Like antibodies (and their equivalents), RNAi agents that can be applied in the present invention are known in the art. Briefly, RNA interference (RNAi) is a biological process in which RNA molecules inhibit gene expression, typically by causing the destruction of specific mRNA molecules. RNAi is now known as precise, efficient, stable and better than antisense technology for gene suppression. Two types of small ribonucleic acid (RNA) molecules—microRNA (miRNA) and small interfering RNA (siRNA)—are central to RNA interference. RNAs are the direct products of genes, and these small RNAs can bind to other specific messenger RNA (mRNA) molecules and either increase or decrease their activity, for example by preventing an mRNA from producing a protein. However, the present invention is not necessarily limited to siRNA and mRNA.

Whenever the term “at least” or “greater than” precedes the first numerical value in a series of two or more numerical values, the term “at least” or “greater than” applies to each one of the numerical values in that series of numerical values.

The term “heterologous” refers to an amino acid or nucleotide sequence that is not naturally found in association with the amino acid or nucleotide sequence with which it is associated.

The term “natural” or “naturally occurring” as applied to an object refers to the fact that the object can be found in nature. For example, a polypeptide or polynucleotide sequence which is present in an organism (including viruses) and can be isolated from a source in nature, and which has not been intentionally modified by man in the laboratory, is naturally occurring.

Whenever the term “no more than” or “less than” precedes the first numerical value in a series of two or more numerical values, the term “no more than” or “less than” applies to each one of the numerical values in that series of numerical values.

The term “polynucleotide” refers to a polymer composed of nucleotide units. Polynucleotides include naturally occurring nucleic acids, such as deoxyribonucleic acid (“DNA”) and ribonucleic acid (“RNA”), as well as nucleic acid analogs. Nucleic acid analogs include those which contain non-naturally occurring bases, nucleotides that engage in linkages with other nucleotides other than the naturally occurring phosphodiester bond, or/and bases attached through linkages other than phosphodiester bonds. Non-limiting examples of nucleotide analogs include phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, e.g., using an automated DNA synthesizer. The term “nucleic acid molecule” typically refers to larger polynucleotides. The term “oligonucleotide” typically refers to shorter polynucleotides. In certain embodiments, an oligonucleotide contains no more than about 50 nucleotides. It is understood that when a nucleotide sequence is represented by a DNA sequence (i.e., A, T, G, C), this also includes an RNA sequence (i.e., A, U, G, C) in which “U” replaces “T”.

The term “polypeptide” refers to a polymer composed of natural or/and unnatural amino acid residues, naturally occurring structural variants thereof, or/and synthetic non-naturally occurring analogs thereof, linked via peptide bonds. Synthetic polypeptides can be synthesized, e.g., using an automated polypeptide synthesizer. Polypeptides can also be produced recombinantly in cells expressing nucleic acid sequences that encode the polypeptides. The term “protein” typically refers to larger polypeptides. The term “peptide” typically refers to shorter polypeptides. In certain embodiments, a peptide contains no more than about 50, 40 or 30 amino acid residues. Polypeptides include antibodies and fragments thereof. Conventional notation is used herein to portray polypeptide sequences: the left-hand end of a polypeptide sequence is the amino (N)-terminus; the right-hand end of a polypeptide sequence is the carboxyl (C)-terminus.

Polypeptides can include one or more modifications that may be made during the course of synthetic or cellular production of the polypeptide, such as one or more post-translational modifications, whether or not the one or more modifications are deliberate. Modifications can include without limitation glycosylation (e.g., N-linked glycosylation and O-linked glycosylation), lipidation, phosphorylation, sulfation, acetylation (e.g., acetylation of the N-terminus), amidation (e.g., amidation of the C-terminus), hydroxylation, methylation, formation of an intramolecular or intermolecular disulfide bond, formation of a lactam between two side chains, formation of pyroglutamate, and ubiquitination. As another example, a polypeptide can be attached to a natural polymer (e.g., a polysaccharide) or a synthetic polymer (e.g., polyethylene glycol [PEG]), lipidated (e.g., acylated with a C₈-C₂₀ acyl group), or labeled with a detectable agent (e.g., a radionuclide, a fluorescent dye or an enzyme). PEGylation can increase the protease resistance, stability and half-life, increase the solubility and reduce the aggregation of the polypeptide.

The term “conservative substitution” refers to substitution of an amino acid in a polypeptide with a functionally, structurally or chemically similar natural or unnatural amino acid. In certain embodiments, the following groups each contain natural amino acids that are conservative substitutions for one another:
1) Glycine (Gly/G), Alanine (Ala/A);
2) Isoleucine (Ile/I), Leucine (Leu/L), Methionine (Met/M), Valine (Val/V);
3) Phenylalanine (Phe/F), Tyrosine (Tyr/Y), Tryptophan (Trp/W);
4) Serine (Ser/S), Threonine (Thr/T), Cysteine (Cys/C);
5) Asparagine (Asn/N), Glutamine (Gln/Q);
6) Aspartic acid (Asp/D), Glutamic acid (Glu/E); and
7) Arginine (Arg/R), Lysine (Lys/K), Histidine (His/H).

In further embodiments, the following groups each contain natural amino acids that are conservative substitutions for one another:
1) non-polar: Ala, Val, Leu, Ile, Met, Pro (proline/P), Phe, Trp;
2) hydrophobic: Val, Leu, Ile, Phe, Tyr, Trp;
3) aliphatic: Ala, Val, Leu, Ile;
4) aromatic: Phe, Tyr, Trp, His;
5) uncharged polar or hydrophilic: Gly, Ala, Pro, Ser, Thr, Cys, Asn, Gln, Tyr (tyrosine may be regarded as a hydrophobic amino acid with a polar side group);
6) aliphatic hydroxyl- or sulfhydryl-containing: Ser, Thr, Cys;
7) amide-containing: Asn, Gln;
8) acidic: Asp, Glu;
9) basic: Lys, Arg, His; and
10) small: Gly, Ala, Ser, Cys.

In other embodiments, amino acids may be grouped as set out below:
1) hydrophobic: Val, Leu, Ile, Met, Phe, Trp, Tyr;
2) aromatic: Phe, Tyr, Trp, His;
3) neutral hydrophilic: Gly, Ala, Pro, Ser, Thr, Cys, Asn, Gln;
4) acidic: Asp, Glu;
5) basic: Lys, Arg, His; and
6) residues that influence backbone orientation: Pro, Gly.

A polypeptide having one or more modifications relative to a parent polypeptide may be called an “analog”, “derivative” or “variant” of the parent polypeptide as appropriate.

The disclosure encompasses pharmaceutically acceptable salts of polypeptides, including those with a positive net charge, those with a negative net charge, and those with no net charge.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition.

The term “stringent hybridization conditions” refers to hybridizing in 50% formamide at 5XSSC at a temperature of 42° C. and washing the filters in 0.2XSSC at 60° C. (1XSSC is 0.15M NaCl, 0.015M sodium citrate.) Stringent hybridization conditions also encompasses low ionic strength and high temperature for washing, for example 0.015 M sodium chloride/0.0015 M sodium citrate/0.1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5XSSC (0.75 M NaCl, 0.075 M sodium citrate), 50 mM sodium phosphate (pH 6.8), 0.1% sodium pyrophosphate, 5X Denhardt's solution, sonicated salmon sperm DNA (50 μg/ml), 0.1% SDS, and 10% dextran sulfate at 42° C., with washes at 42° C. in 0.2XSSC (sodium chloride/sodium citrate) and 50% formamide at 55° C., followed by a high-stringency wash consisting of 0.1XSSC containing EDTA at 55° C.

The term “subject” refers to an animal, including but not limited to a mammal, such as a primate (e.g., a human, a chimpanzee or a monkey), a rodent (e.g., a rat, a mouse, a guinea pig, a gerbil or a hamster), a lagomorph (e.g., a rabbit), a swine (e.g., a pig), an equine (e.g., a horse), a canine (e.g., a dog) or a feline (e.g., a cat).

The term “substantially homologous” or “substantially identical” in the context of two polypeptides or polynucleotides refers to two or more sequences or subsequences that have at least about 50%, 60%, 65%, 70%, 75%, 80%, 85%, 90%, 95%, 96%, 97%, 98% or 99% amino acid or nucleic acid residue identity, when compared and aligned for maximum correspondence, as measured using a sequence comparison algorithm or by visual inspection. The terms “substantially homologous” or “substantially identical” can mean at least about 70% amino acid or nucleic acid residue identity. The term “substantially homologous” or “substantially identical” can mean at least about 85% amino acid or nucleic acid residue identity. The substantial homology or identity can exist over a region of the sequences that is at least about 20, 30, 40, 50, 100, 150 or 200 residues in length. The sequences can be substantially homologous or identical over the entire length of either or both comparison biopolymers.

Optimal alignment of sequences for comparison can be conducted, e.g., by the local homology algorithm of Smith and Waterman, Adv. Appl. Math., 2:482 (1981); by the homology alignment algorithm of Needleman and Wunsch, J. Mol. Biol., 48:443 (1970); by the search for similarity method of Pearson and Lipman, Proc. Natl. Acad. Sci. USA, 85:2444 (1988); by computerized implementations of these algorithms (e.g., GAP, BESTFIT, FASTA, and TFASTA in the Wisconsin Genetics Software Package, Genetics Computer Group, Madison, Wis.); or by visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle, J. Mol. Evol., 35:351-360 (1987). The method used is similar to the method described by Higgins and Sharp, CABIOS, 5:151-153 (1989). The program can align up to about 300 sequences, each having a maximum length of about 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps. Another algorithm that is useful for generating multiple alignments of sequences is Clustal W (see, e.g., Thompson et al., Nucleic Acids Research, 22:4673-4680 [1994]).

Another example of an algorithm that is suitable for determining percent sequence identity and sequence similarity is the BLAST algorithm, which is described in Altschul et al., J. Mol. Biol., 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults, e.g., a wordlength (W) of 11, an expectation (E) of 10, M=5, N=−4, and a comparison of both strands. For amino acid sequences, the BLASTP program uses as defaults, e.g., a wordlength (W) of 3, an expectation (E) of 10, and the BLOSUM62 scoring matrix (see Henikoff and Henikoff, Proc. Natl. Acad. Sci. USA, 89:10915 [1989]).

In addition to calculating percent sequence identity, the BLAST algorithm also performs a statistical analysis of the similarity between two sequences (see, e.g., Karlin and Altschul, Proc. Natl. Acad. Sci. USA, 90:5873-5787 [1993]). One measure of similarity provided by the BLAST algorithm is the smallest sum probability [P(N)], which provides an indication of the probability by which a match between two nucleotide or amino acid sequences would occur by chance. In certain embodiments, a polynucleotide is considered similar to a reference sequence if the smallest sum probability in a comparison of the test polynucleotide to the reference polynucleotide is less than about 0.1, 0.01 or 0.001.

In some aspects, a polypeptide is substantially homologous or identical to a second polypeptide if the two polypeptides differ only by conservative amino acid substitutions. In certain embodiments, two nucleic acid sequences are substantially homologous or identical if the two polynucleotides hybridize to each other under stringent conditions, or under highly stringent conditions, as described herein.

Disclosed herein is a method of treating a Group 2 innate lymphoid cell (ILC2) mediated disease comprising administering to a subject in need thereof an effective amount of a killer cell lectin-like receptor G1 (KLRG1)/ligand binding agent.

Further disclosed herein is a method of treating a Group 2 innate lymphoid cell (ILC2) mediated disease in a subject in need thereof comprising administering to a subject in need thereof an effective amount of a KLRG1 antagonist or a KLGR1 ligand antagonist.

The methods disclosed herein can use a combination of the KLRG1/ligand binding agent or a KLRG1 antagonist/ligand antagonist and one or more additional Active Pharmaceutical Ingredient (API). Accordingly, the invention can increase the efficacy of, and/or decrease undesired side effects from, the KLRG1/ligand binding agent or a KLRG1 antagonist/ligand antagonist.

The methods further comprises administering to the subject an effective amount of an antibiotic agent, an antiviral agent, an anti-fungal agent, or an analgesic.

Exemplary antibiotic agents may include but are not limited to aminoglycoside; amikacin; gentamicin; kanamycin; neomycin; netilmicin; steptomycin; tobramycin; ansamycins; geldanamycin; herbimycin; carbacephem; loracarbef; carbacepenem; ertapenem; doripenem; imipenem/cilastatin; meropenem; cephalosporin; cefadroxil; cefazolin; cefalotin or cefalothin; cefalexin; cefaclor; cefamandole; cefoxitin; cefprozil; cefuroxime; cefixime; cefdinir; cefditoren; cefoperazone; cefotaxime; cefpodoxime; ceftazidime; ceftibuten; ceftizoxime; ceftriaxone; cefepime; ceftobiprole; glycopeptide; teicoplanin; vancomycin; macrolides; azithromycin; clarithromycin; dirithromycin; erythromicin; roxithromycin; troleandomycin; telithromycin; spectinomycin; monobactam; aztreonam; penicillins; amoxicillin; ampicillin; azlocillin; carbenicillin; cloxacillin; dicloxacillin; flucloxacillin; mezlocillin; meticillin; nafcillin; oxacillin; penicillin, piperacillin, ticarcillin; bacitracin; colistin; polymyxin B; quinolone; ciprofloxacin; enoxacin; gatifloxacin; levofloxacin; lomefloxacin; moxifloxacin; norfloxacin; ofloxacin; trovafloxacin; sulfonamide; mafenide; prontosil (archaic); sulfacetamide; sulfamethizole; sufanilimide (archaic); sulfasalazine; sulfisoxazole; trimethoprim; trimethoprim-sulfamethoxazole (co-trimoxazole) (TMP-SMX); tetracycline; demeclocycline; doxycycline; minocycline; oxytetracycline; tetracycline; arsphenamine; chloramphenicol; clindamycin; lincomycin; ethambutol; fosfomycin; fusidic acid; furazolidone; isoniazid; linezolid; metronidazole; mupirocin; nitrofuantoin; platensimycin; polymyxin, purazinamide; quinupristin/dalfopristin; rifampin or rifampicin; and timidazole.

Exemplary antiviral agents may include, but are not limited to thiosemicarbazone; metisazone; nucleoside and/or nucleotide; acyclovir; idoxuridine; vidarabine; ribavirin; ganciclovir; famciclovir; valaciclovir; cidofovir; penciclovir; valganciclovir; brivudine; ribavirin, cyclic amines; rimantadine; tromantadine; phosphonic acid derivative; foscamet; fosfonet; protease inhibitor; saquinavir; indinavir; ritonavir; nelfinavir; amprenavir; lopinavir; fosamprenavir; atazanavir; tipranavir; nucleoside and nucleotide reverse transcriptase inhibitor; zidovudine; didanosine; zalcitabine; stavudine; lamivudine; abacavir; tenofovir disoproxil; adefovir dipivoxil; emtricitabine; entecavir; non-nucleoside reverse transcriptase inhibitor; nevirapine; delavirdine; efavirenz; neuraminidase inhibitor; zanamivir; oseltamivir; moroxydine; inosine pranobex; pleconaril; and enfuvirtide.

Exemplary anti-fungal agents may include but are not limited to allylamine; terbinafine; antimetabolite; flucytosine; azole; fluconazole; itraconazole; ketoconazole; ravuconazole; posaconazole; voriconazole; glucan synthesis inhibitor; caspofungin; micafungin; anidulafungin; polyenes; amphotericin B; amphotericin B Colloidal Dispersion (ABCD); and griseofulvin.

Exemplary analgesics may include, but are not limited to opiate derivative, codeine, meperidine, methadone, and morphine.

In various aspects, the KLRG1/ligand binding agent or KLRG1 antagonist/ligand antagonist is prepared as a pharmaceutical composition, for example as a pharmaceutical composition for use as a medicament. In various aspects, the pharmaceutical composition is for use as a therapeutic. In various aspects, the pharmaceutical composition can include one or more antibiotic, antivirus, anti-diabetes, anti-hypertension, anti-fungal, or analgesic.

One skilled in the art can formulate the KLRG1/ligand binding agent or KLRG1 antagonist/ligand antagonist as a pharmaceutical composition according to known methods.

Pharmaceutical compositions can include a carrier. “Carriers” as used herein can include pharmaceutically acceptable carriers, excipients, or stabilizers which are nontoxic (or relatively non-toxic) to the cell or subject being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In various aspects, the KLRG1/ligand binding agent or KLRG1 antagonist/ligand antagonist is comprised in an injectable formulation, for example, a subcutaneous, intravenous, intramuscular, intrathecal or intraperitoneal injection formulation. Injectable formulations can be aqueous solutions, for example in physiologically compatible buffers such as Hanks' solution, Ringer's solution, or physiological saline buffer. The injectable formulation can contain formulatory agents such as suspending, stabilizing and/or dispersing agents. Alternatively, the KLRG1/ligand binding agent or KLRG1 antagonist/ligand antagonist can be in a dried or powder form for constitution with a suitable vehicle, e.g., sterile pyrogen-free water, before use.

The term “therapeutically effective amount” refers to an amount of a compound that, when administered to a subject, is sufficient to prevent, reduce the risk of developing, delay the onset of, slow the progression or cause regression of the medical condition being treated, or to alleviate to some extent the medical condition or one or more symptoms or complications of that condition. The term “therapeutically effective amount” also refers to an amount of a compound that is sufficient to elicit the biological or medical response of a cell, tissue, organ, system, animal or human which is sought by a researcher, veterinarian, medical doctor or clinician. As such, in various embodiments, the term “effective amount” is a concentration or amount of the KLRG1/ligand binding agent or KLRG1 antagonist/ligand antagonist which results in achieving a particular stated purpose. An “effective amount” of a KLRG1/ligand binding agent or KLRG1 antagonist/ligand antagonist can be determined empirically. Furthermore, a “therapeutically effective amount” is a concentration or amount of a KLRG1/ligand binding agent or KLRG1 antagonist/ligand antagonist which is effective for achieving a stated therapeutic effect. This amount can also be determined empirically.

The terms “treat”, “treating” and “treatment” include alleviating, ameliorating or abrogating a medical condition or one or more symptoms or complications associated with the condition, and alleviating, ameliorating or eradicating one or more causes of the condition. The terms “prevent”, “preventing” and “prevention” include precluding, reducing the risk of developing and delaying the onset of a medical condition or one or more symptoms or complications associated with the condition.

“Administration” and “treatment,” as it applies to an animal, human, experimental subject, cell, tissue, organ, or biological fluid, can include contacting an exogenous pharmaceutical, therapeutic agent, diagnostic agent, or composition to the animal, human, subject, cell, tissue, organ, or biological fluid. “Administration” and “treatment” include in vivo, as well as in some embodiments in vitro or ex vivo treatments.

The methods disclosed herein comprise administering the KLRG1/ligand binding agent or KLRG1 antagonist/ligand antagonist according to any of the aspects disclosed herein, or the pharmaceutical composition according to any of the aspects disclosed herein, to a subject in need thereof. In some aspects, the subject is a human. In various aspects, the methods disclosed herein are carried out in vivo (e.g., as opposed to ex vivo).

The following experimental results are provided for purposes of illustration and are not intended to limit the scope of the invention.

### EXAMPLES

### Example 1. Generation of Anti-KLRG1 Hybridoma

Three to five mice are immunized by subcutaneous injection of immunogen on Day 1, Day 22 and Day 43. Injections are emulsions in Complete Freund's Adjuvant (CFA) or Incomplete Freund's Adjuvant (IFA). The first injection is 100 μg of antigen in CFA. The booster injections are 50 μg of antigen in IFA. Serum is drawn from each of the mice on Day 0, 29, 50, and assayed to determine seroconversion (antibody production). Each animal demonstrating seroconversion is boosted a 3rd time on Day 63 with 100 μg of soluble antigen injected intravenously. The animals are sacrificed 3 days later to harvest the splenocytes. Alternatively, each mouse is injected of 10 μg antigen mixed with adjuvant at footpad of two hind limbs once a week for 6 consecutive weeks. Serum samples are collected and specific antibody titers are determined by Elisa after the 6th injection. The mice with the highest titer are boosted with antigen (no adjuvant) three days in a row prior to collection of lymph nodes and splenocytes.

The B cells are fused with myeloma cells at 1:1 ratio facilitated with 35% polyethylene glycol (PEG) (mw, 1500), and then plated in 96-w plates at 2.5×10⁴ myeloma cells/100 μl/well in growth media without hypoxanthine-aminopterin-thymidine (HAT). Next day, 100 μl/well of growth media plus 2× HAT are added into the cells.

After hybridoma clones grow out in —10 days, the supernatant are screened for anti-KLRG1 antibody by Elisa. The antigen is coated in the Elisa plate. After blocking followed by incubation of the hybridoma supernatant, the bound antibody are detected with anti-mouse IgG HRP conjugate.

### Example 2. Generation of Phage-Derived, Human Anti-KLRG1 Antibodies

A human phagemid library with 1.2×10¹⁰ independent clones is panned against recombinant soluble KLRG1. Typically, one mL of unamplified human Fab phage display library is panned in three sequential rounds against 100 nM, 40 nM, and 10 nM of biotinylated antigen bound to streptavidin magnetic particles. Bead-antigen-phage hybridizations are washed and eluted with increasing stringency through the rounds of enrichment until after 3 rounds, KLRG1-specific phage are competitively eluted from the antigen-beads by 20× molar excess (200 nM) of free antigen in solution. Phage that remain bound to antigen post competitive elution are recovered in 100 mM triethylamine. TG1 cells are infected with the two phage populations enriched for binders and plated. Individual clones are picked and screened against KLRG1 by ELISA either with phage directly or with expressed Fab.

Additional panning rounds may further enrich for populations of KLRG1-specific binders, while the number of clones screened for binding can be increased from several hundred to several thousand. Finally, the library may be re-screened adjusting the panning parameters based on prior results (antigen concentrations, using competitive elutions earlier in the panning process, etc.) leading to a different sampling of the library and new sets of anti-KLRG1 clones.

Positive anti-KLRG1 clones are re-expressed to confirm original binding by ELISA, sequenced and rank-ordered by estimated affinity and clone sequence relationships. Clones that reconfirm binding, are reconstituted as IgG, and expressed as such for all downstream antibody and biological assay based characterizations.

### Example 3. Anti-KLRG1 Treatment to Prevent Airway Hyperresponsiveness

Mice (strain Yetcre13) were infected with 500 Nippostrongylus brasihensis stage 3 larvae. On days 2, 3, and 4 the mice were given intravenous 100 ug of anti-KLRG1 clone 2F1. The following day the mice were euthanized and their lungs harvested for analysis of ILC2 cells and eosinophils.

| | | | | |
|---|---|---|---|---|
| D0 | D2 | D3 | D4 | D5 |
| 500L3 Nb | aKLRG1 | aKLRG1 | aKLRG1 | sac |

Rationale for the mouse strain: Yetcre13 mice are C57BL/6 mice that express the yellow fluorescent protein (YFP) when the cytokine IL-13 is transcribed. This mouse strain is useful in detecting IL-13-producing cells by flow cytometry.

Rationale for using Nippostrongylus brasihensis: “Nippo” was chosen because it initiates a very robust pulmonary type-2 immune response with pathology similar to human allergic asthma. The lifecycle of Nippo is such that the larvae migrate through epithelium into vasculature (we inject the larvae subcutaneously in the hind flank). The larvae get trapped in the capillary beds of the lung where they migrate into the lung parenchyma and mature into stage 4 larvae. They then migrate to the alveolar airspace, causing significant tissue damage/hemorrhage. They then ascend the trachea and are swallowed where they mature again and reside in the small intestine. In a mouse with an intact immune system they are cleared 8-9 days after infection. Important to our proposal, the response to Nippo is characterized by mucus production, small muscle contractility, and airway constriction. These processes are mediated by lymphocyte effector function, eosinophilia, and ILC2 function.

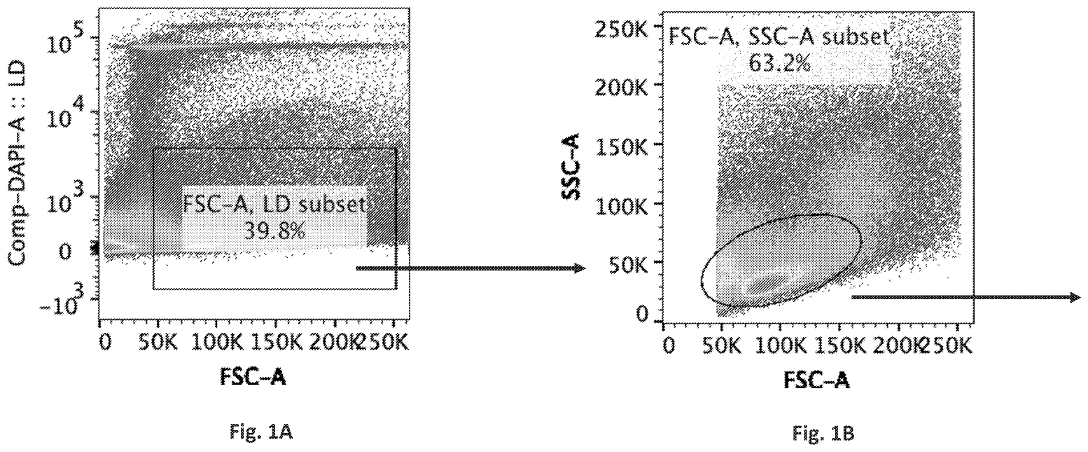

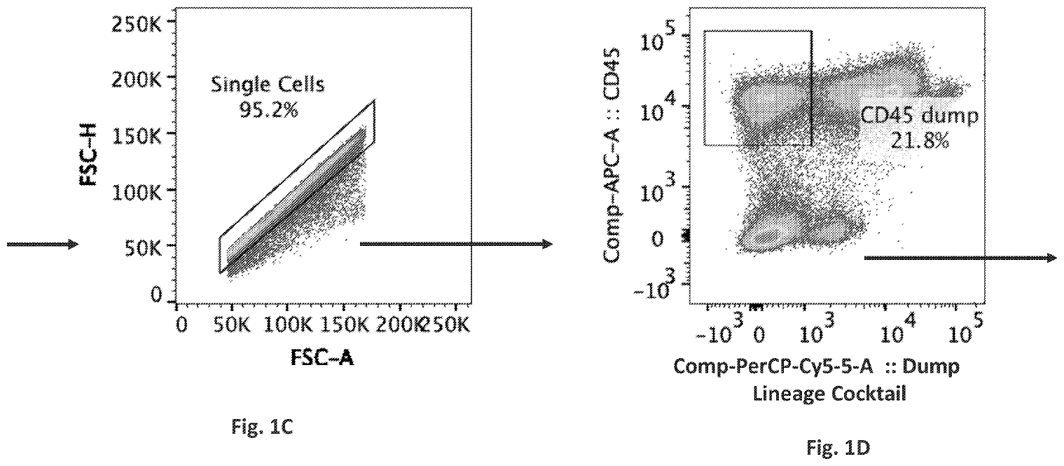

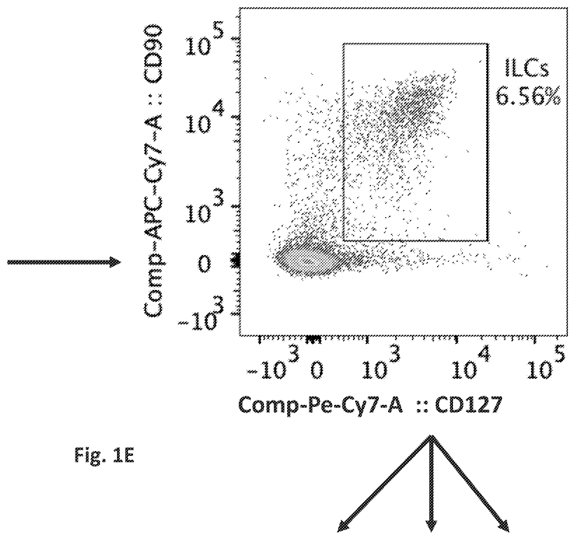

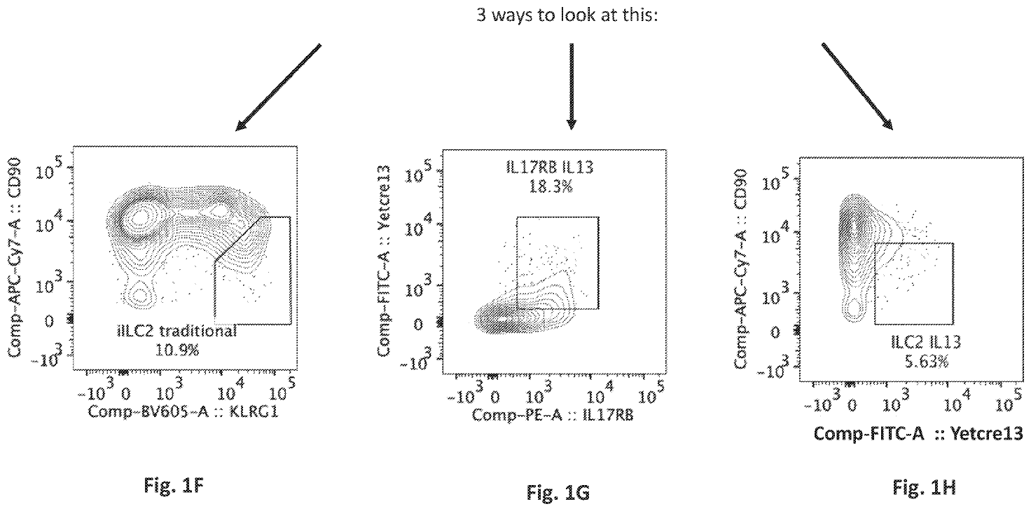

Using Flow cytometry, inflammatory ILC2s (iILC2s) can be identified in three different ways. First, live, small, single cells are looked at and then are gated CD45 positive and Lineage negative. The lineage antibody cocktail includes CD3, CD4, CD11b, CD11c, B220, NK1.1, FcεR1a, Gr-1, γδTCR, TCRβ, and Ter119. Then ILCs are gated by CD90 and CD127 positive. From there, iILC2s can be characterized by the following: traditionally by CD90 intermediate and KLRG1 high; IL-13 producing IL-25R expressing cells; or CD90 intermediate CD25R positive cells. (FIGS. 1A-1H)

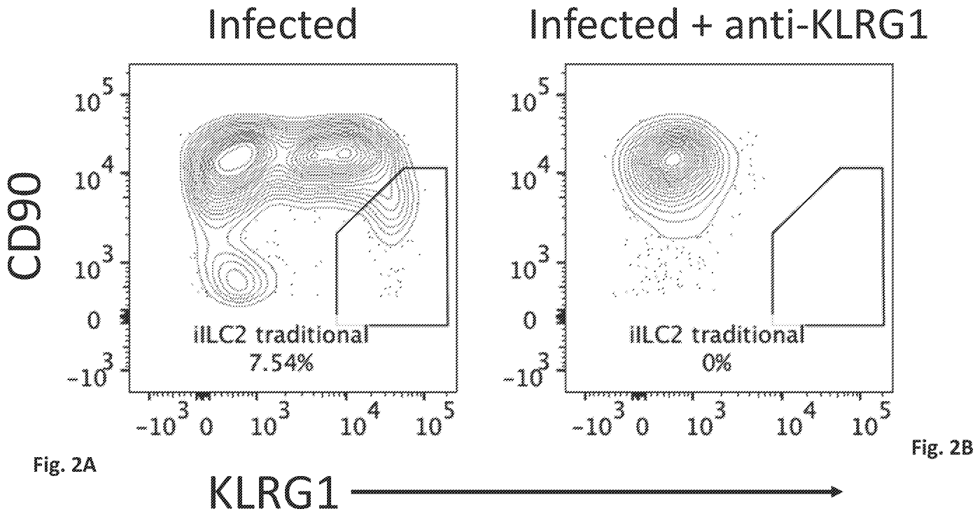

The anti-KLRG1 IV treatment was validated by fully saturating and binding all surface KLRG1 on ILCs. The flow plots in FIGS. 2A-2B show a black polygon that indicates the presence of iILC2s in infected mice but not in mice given anti-KLRG1. Because the same antibody clone was used for flow cytometry staining, there are no available KLRG1 epitopes for the flow cytometry antibody to bind in mice that were given in vivo anti-KLRG1.

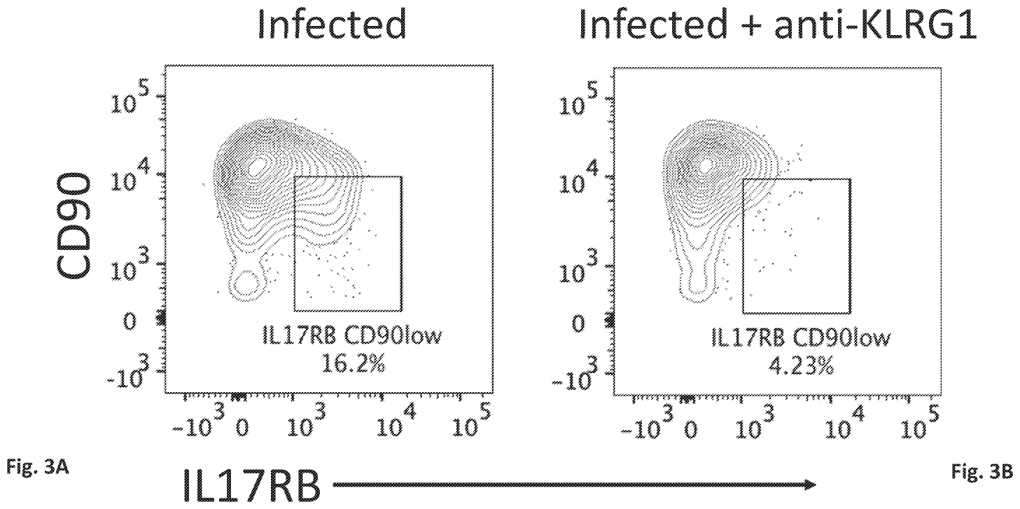

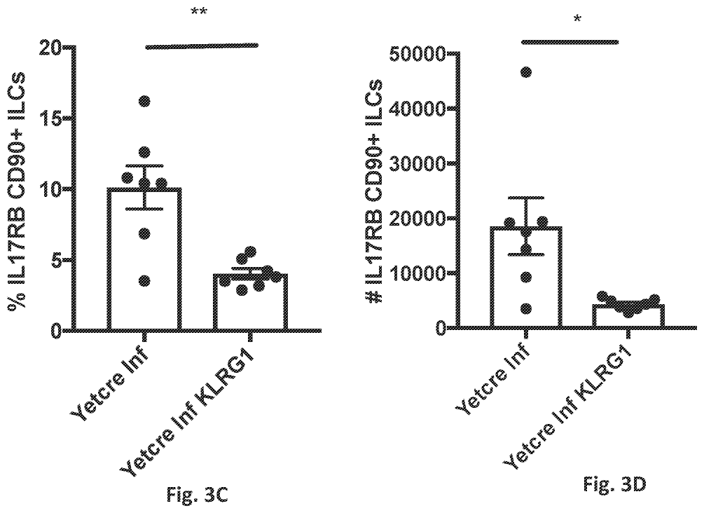

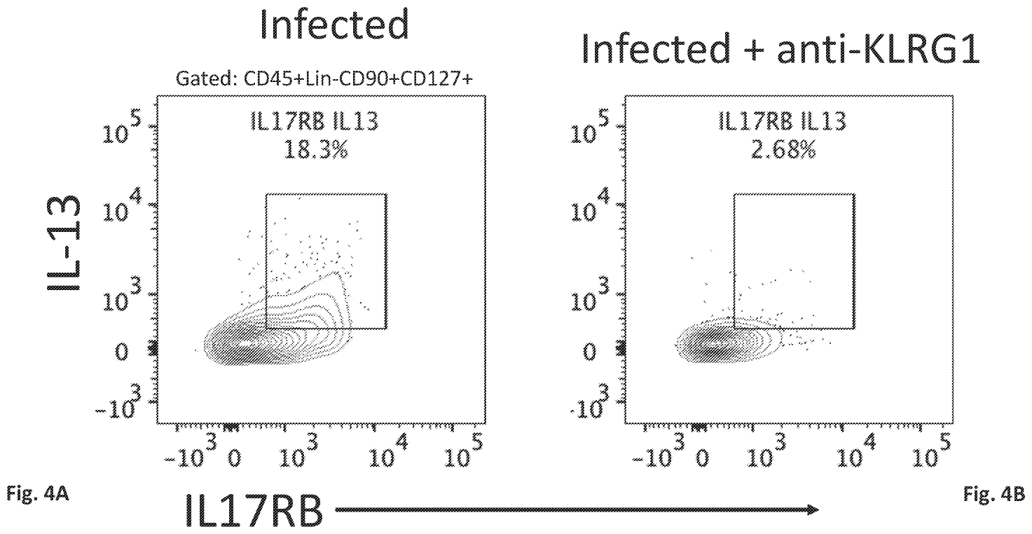

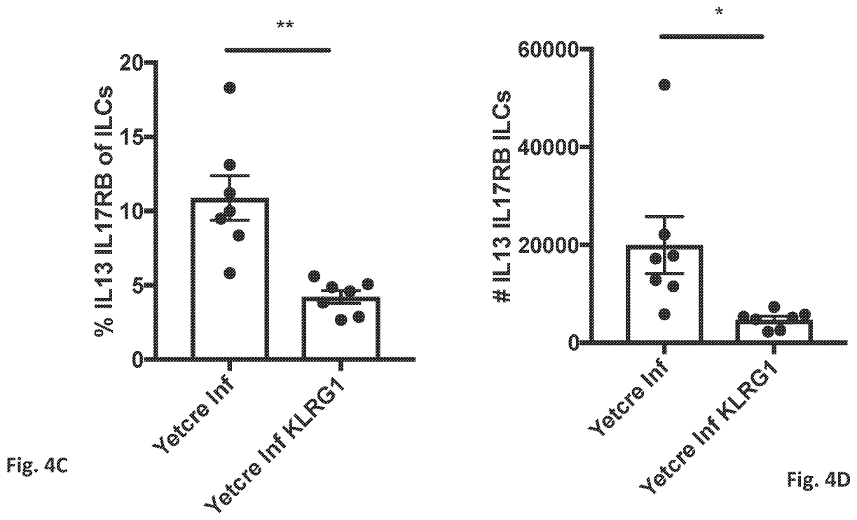

Treatment with anti-KLRG1 was able to significantly reduce the recruitment of iILC2s to the lung compared to infected mice not given anti-KLRG1 treatment. FIGS. 3A-3D show the significant reduction in IL-17RB (IL25-receptor) positive CD90int/low iILC2s upon anti-KLRG1 treatment. FIGS. 4A-4D show that anti-KLRG1 treatment significantly reduces IL-13-producing IL25-responsive ILC2s in the lung at day 5.

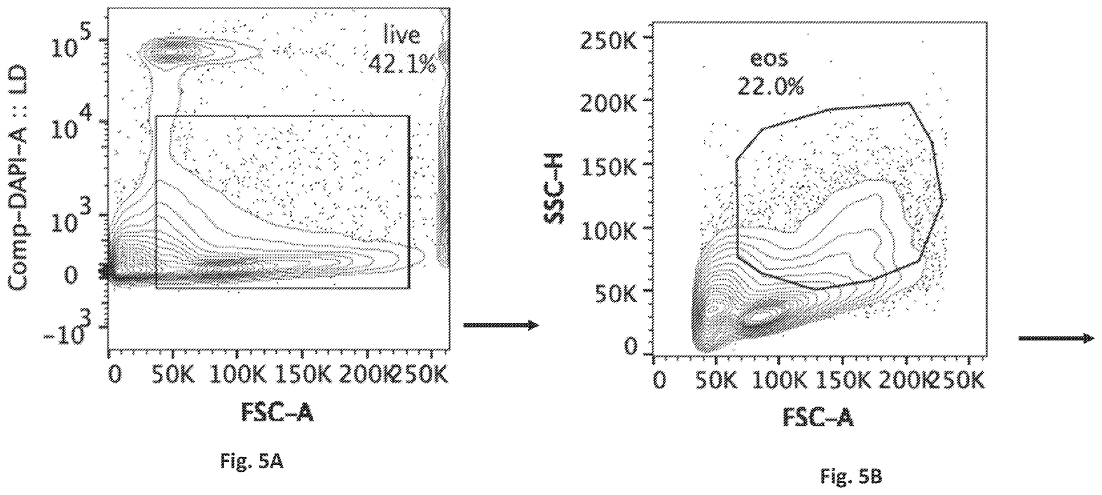

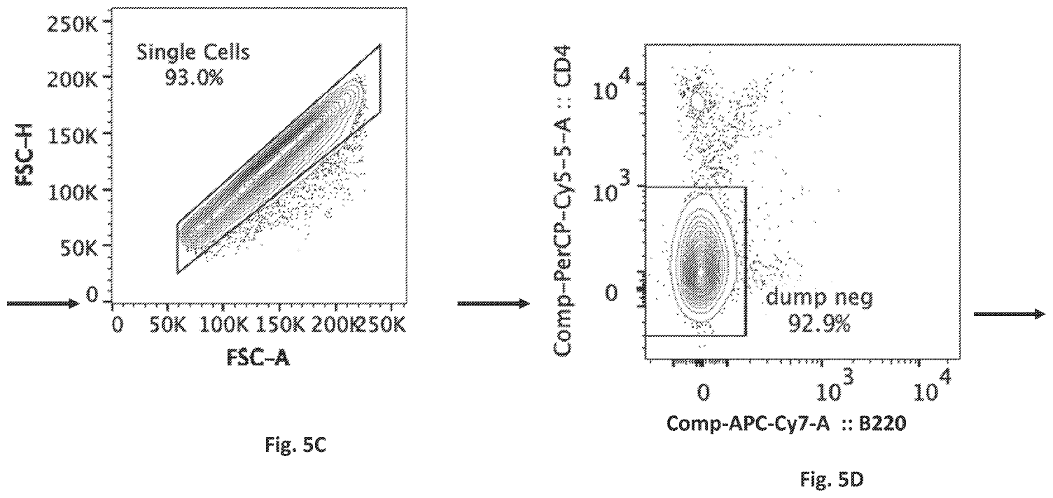

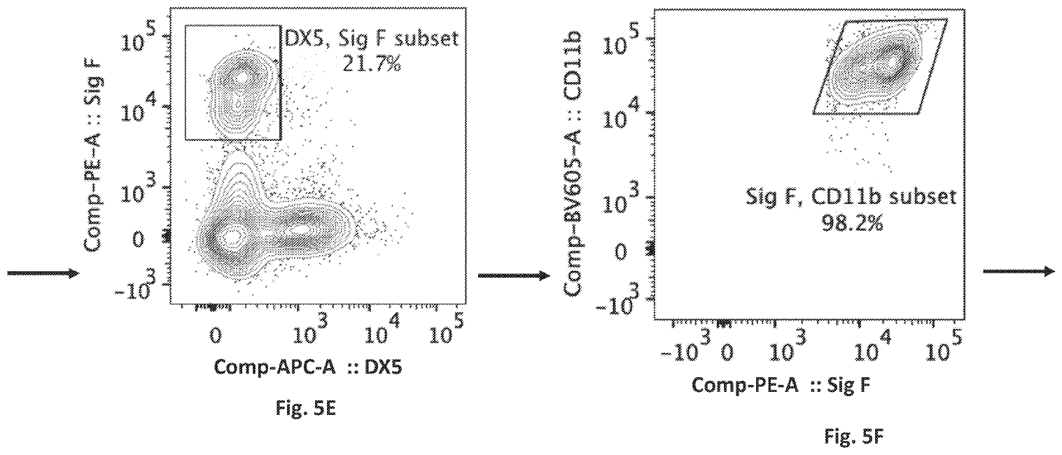

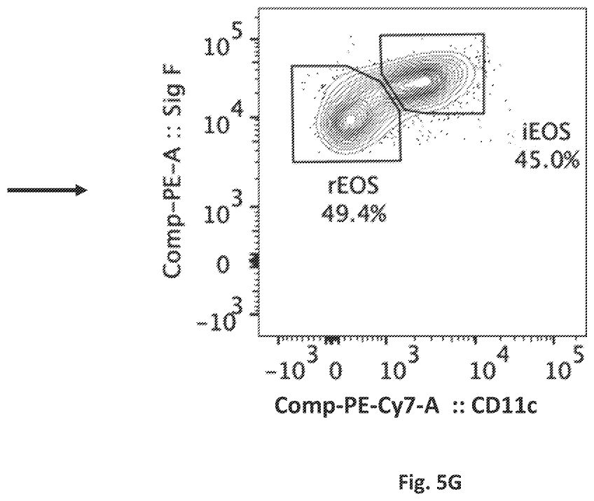

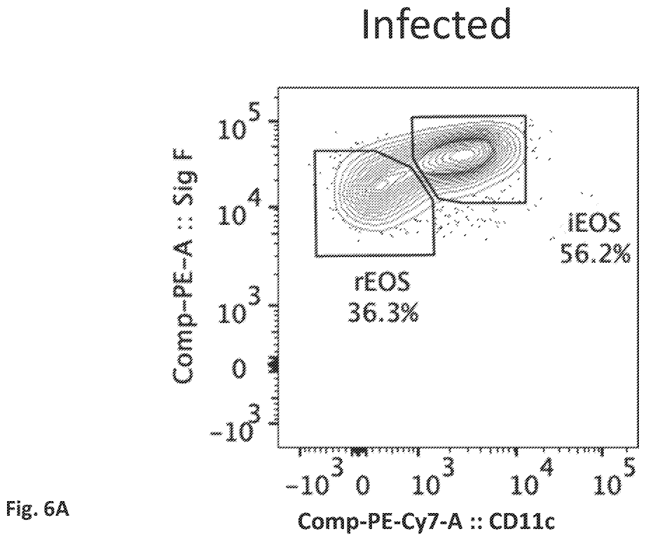

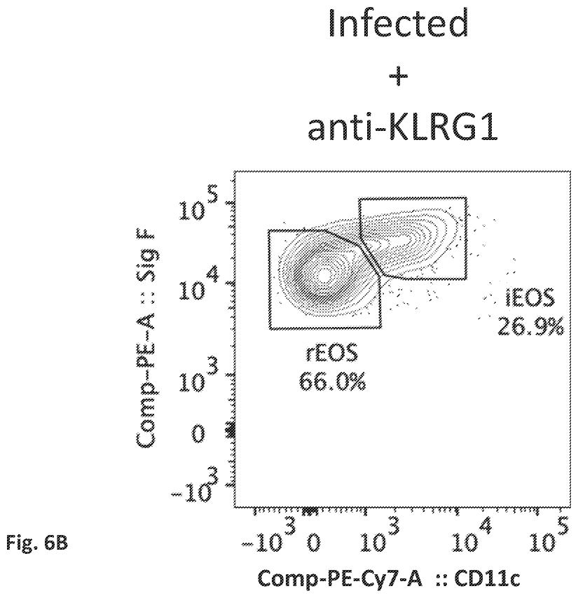

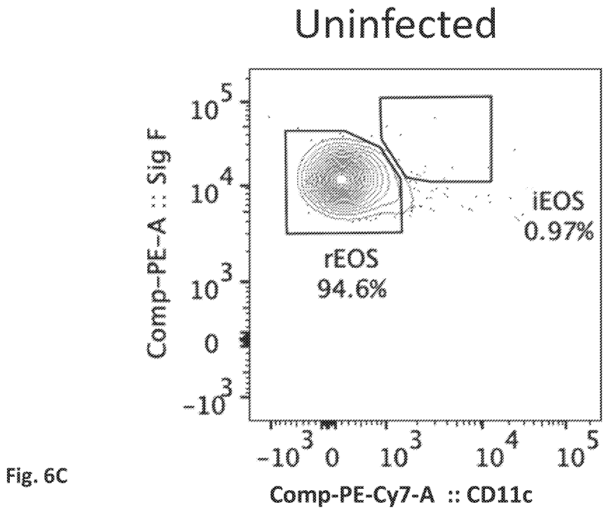

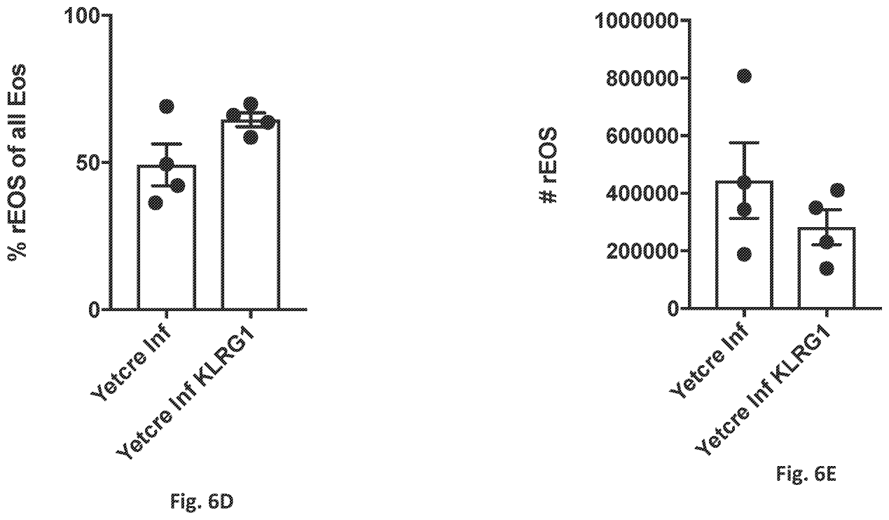

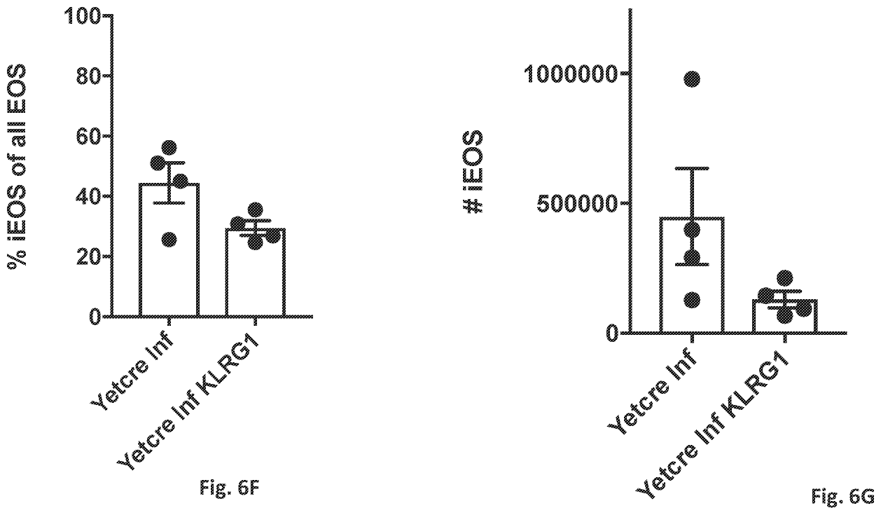

Eosinophil populations were assessed in anti-KLRG1 treatment as eosinophils are known to exacerbate allergic disease. Moreover, IL-5-producing ILC2s can directly aid in bone marrow egress of eosinophils which is required for pulmonary eosinophil accumulation. Eosinophils are identified in the lung by flow cytometry analysis of live, side-scatter high, singlet cells that do not express CD4 or B220 and that are Siglec-F high but DX5 negative. These cells also express CD11b. CD11c can be used to subset resident parenchymal eosinophils (rEOS) from recruited inflammatory airway eosinophils (iEOS). (FIGS. 5A-G) Anti-KLRG1 treatment resulted in the reduction of iEOS (FIGS. 6A-6G). The ability of anti-KLRG1 to reduce iEOS is surprising and provides support that anti-KLRG1 treatment can result in reduced pulmonary allergic disease.

All of the documents cited herein are incorporated herein by reference.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

What is claimed:

1. A method of treating a Group 2 innate lymphoid cell (ILC2) mediated disease comprising administering to a subject in need thereof an effective amount of a killer cell lectin-like receptor G1 (KLRG1) binding agent or KLRG1 ligand binding agent.

2. The method of claim 1, wherein the binding agent is an antibody or antigen binding fragment thereof, an antibody mimetic or an antibody mimetic that binds the extracellular domain of human KLRG1.

3. The method of claim 2, wherein the antibody or antigen binding fragment thereof, or antibody mimetic comprises a human or humanized antibody.

4. The method of claim 2, wherein the antibody or antigen binding fragment thereof, or antibody mimetic comprises:
(a) full length antibody Fab portion that binds KLRG1;
(b) a full length antibody Fab portion that binds E-cadherin;
(c) a full length antibody Fab portion that binds N-cadherin;
(d) a full length antibody Fab portion that binds R-cadherin;
(e) a fusion protein E-cadherin/Fc;
(f) a fusion protein R-cadherin/Fc;
(g) a fusion protein N-cadherin/Fc;
(h) a chimeric antigen receptor; or
(i) a multi-specific antibody.

5. The method of claim 4, wherein the multi-specific antibody comprises a bispecific or tri-specific antibody.

6. The method of claim 1, wherein the KLRG1 binding agent binds KLRG1.

7. The method of claim 6, wherein the KLRG1 is the extracellular domain of human KLRG1.

8. The method of claim 1, wherein the KLRG1 ligand binding agent binds KLRG1 ligand.

9. The method of claim 8, wherein the KLRG1 ligand is human E-cadherin, N-cadherin, or R-cadherin.

10. The method of claim 1, wherein the binding agent binds KLRG1 and/or cadherin at a KLRG1/cadherin binding site.

11. The method of claim 1, wherein the ILC2 mediated disease is selected from the group consisting of asthma, atopic dermatitis, allergic airway disease, airway hyperreactivity, idiopathic pulmonary fibrosis, chronic rhinosinusitis, respiratory syncytial virus induced airway inflammation, chronic obstructive pulmonary disease (COPD), liver fibrosis, viral hepatitis, atherosclerosis, multiple sclerosis, and Eosinophilic esophagitis.

12. The method of claim 11, wherein the asthma is fungal asthma or viral asthma from influenza A.

13. The method of claim 11, wherein the allergic airway disease is allergic rhinitis, allergi dermatitis or allergic inflammation from ragweed, house dust mite, or protease allergens.

14. A method of treating a Group 2 innate lymphoid cell (ILC2) mediated disease in a subject in need thereof comprising administering to a subject in need thereof an effective amount of a KLRG1 antagonist or a KLGR1 ligand antagonist.

15. The method of claim 14, wherein the KLRG1 antagonist comprises
(i) KLRG1 binding agent or KLRG1 ligand binding agent; or
(ii) an RNAi agent that suppresses KLRG1 expression; or
(iii) an RNAi agent that suppresses the KLRG1 ligand expression.

16. The method of claim 15, wherein the KLRG1 antagonist disrupts KLRG1 signaling, thereby activating CD8⁺ cytotoxic T and/or NK cells.

17. The method of claim 15, wherein the KLRG1 antagonist binds the extracellular domain of human KLRG1 or binds a KLRG1 ligand.

18. The method of claim 15, wherein the KLRG1 antagonist comprises an antibody or antigen binding fragment.

19. The method of claim 15, wherein the KLRG1 antagonist comprises an Fc-cadherin fusion protein.

20. The method of claim 18, wherein the antibody is monoclonal.

21. The method of claim 18, wherein the antibody is human or humanized.

22. The method of claim 15, wherein the KLRG1 antagonist comprises a binding agent that blocks or competes with KLRG1-ligand binding.

23. The method of claim 15, wherein the KLRG1 antagonist comprises a binding agent that cross reacts with the extracellular domains of human KLRG1, or the human KLRG1 ligand.

24. The method of claim 15, wherein the KLRG1 antagonist comprises a binding agent that binds to KLRG1 and that is not a mouse antibody.

25. The method of claim 15, wherein the KLRG1 antagonist comprises an RNAi agent.

26. The method of claim 15, wherein the KLRG1 antagonist comprises an siRNA or an mRNA.

27. The method of claim 14, wherein the ILC2 mediated disease is selected from the group consisting of asthma, atopic dermatitis, allergic airway disease, airway hyperreactivity, idiopathic pulmonary fibrosis, chronic rhinosinusitis, respiratory syncytial virus induced airway inflammation, chronic obstructive pulmonary disease (COPD), liver fibrosis, viral hepatitis, atherosclerosis, multiple sclerosis, and Eosinophilic esophagitis.

28. The method of claim 27, wherein the asthma is fungal asthma or viral asthma from influenza A.

29. The method of claim 27, wherein the allergic airway disease is allergic rhinitis, allergic dermatitis or allergic inflammation from ragweed, house dust mite, or protease allergens.

30. A killer cell lectin-like receptor G1 (KLRG1) antagonist comprising:
(i) a killer cell lectin-like receptor G1 (KLRG1)/ligand binding agent; or
(ii) an RNAi agent that suppresses KLRG1 expression.

31. The KLRG1 antagonist of claim 30, wherein the antagonist disrupts KLRG1 signaling, thereby activating CD8⁺ cytotoxic T and/or NK cells.

32. The KLRG1 antagonist of claim 30, wherein the antagonist binds the extracellular domain of human KLRG1 or binds a KLRG1 ligand.

33. The KLRG1 antagonist of claim 30, comprising an antibody or antigen binding fragment.

34. The KLRG1 antagonist of claim 30, comprising an Fc-cadherin fusion protein.

35. The KLRG1 antagonist of claim 33, wherein the antibody is monoclonal.

36. The KLRG1 antagonist of claim 33, wherein the antibody is human or humanized.

37. An isolated KLRG1 monoclonal antibody.

38. A nucleic acid polymer encoding the monoclonal antibody of claim 37.
